(19) United States
(12) Patent Application Publication
(10) Pub. No.: US 2025/0271381 A1
(43) Pub. Date: Aug. 28, 2025

(54) Sensor arrangement for an apparatus for additive manufacturing, and apparatus therefor, and measuring method on the basis thereof

(71) Applicant: EOS GMBH ELECTRO OPTICAL SYSTEMS, Krailling (DE)

(72) Inventors: Florian Bayer, Unterhaching (DE); Sjoerd Fruijtier, München (DE); Peter Steinberg, Pflach (AT); Julian Erath, München (DE)

(21) Appl. No.: 18/690,664

(22) PCT Filed: Sep. 13, 2022
(86) PCT No.: PCT/EP2022/075449

§ 371 Date: Aug. 8, 2024

(30) Foreign Application Priority Data
Sep. 21, 2021 (DE) 10 2021 124 450.1

(51) Int. Cl. G01N 27/12; B22F 10/322; B22F 12/90; B33Y 50/02; G01N 33/00

(52) U.S. Cl. CPC G01N 27/12; B22F 10/322; B22F 12/90; B33Y 50/02; G01N 33/0027

(57) ABSTRACT

Disclosed is a sensor arrangement, manufacturing apparatus, and measurement method for an additive manufacture apparatus The sensor arrangement includes a sensor module which is configured to detect oxygen molecules in a gas sample permeating into the sensor module and to generate an electrical sensor signal based on the quantity of the oxygen molecules, a control module which is configured, by means of a comparison of the sensor signal or a variable derived from the sensor signal with a specified threshold value, to determine whether the sensor module is measuring outside a predetermined action range and if this is the case, to generate a control signal which is configured to initiate a predetermined countermeasure which is intended to modify the conditions in the apparatus in a manner such that the sensor module is again measuring in the action range.

The invention relates to a sensor arrangement for an apparatus for the additive manufacture of a component in a manufacturing process in which build material, preferably comprising a metal powder, is consolidated layer by layer on a construction area in a processing area by means of irradiation of the build material with at least one energy beam, as well as to an apparatus of this type and a measurement method with a sensor arrangement of this type.

Additive manufacturing processes are becoming more and more relevant to the production of prototypes and recently also in mass production. In general, the term “additive manufacturing processes” should be understood to mean those manufacturing processes in which, generally on the basis of digital 3D construction data, a manufactured product (also referred to below as a “component”) is constructed by depositing material (the “build material”). The construction is usually layer by layer, but not necessarily so. A synonym for additive manufacture which is also often employed is the term “3D printing”; the production of models, samples and prototypes with additive manufacturing processes is often termed “rapid prototyping”; the production of tools is often termed “rapid tooling” and the flexible production of mass produced components is often termed “rapid manufacturing”. A central point is the selective consolidation of the build material, wherein in many manufacturing processes, this consolidation may be carried out with the aid of an irradiation with radiant energy, for example electromagnetic radiation, in particular light radiation and/or thermal radiation, but if appropriate also with particle beams such as electron beams, for example. Examples of processes operating by irradiation are “selective laser sintering” or “selective laser melting”. In this regard, thin layers of a mainly powdered build material are repetitively applied one on top of the other. In each layer, the build material is selectively consolidated by spatially limited irradiation of the points which are intended to belong to the component to be produced after manufacture, in a “welding process” in which the grains of powder of the build material are partially or completely melted with the aid of the localized energy introduced at this point by the radiation. After cooling, these grains of powder are then consolidated together to form a solid body. In this regard, usually, the energy beam is guided along consolidation tracks over the construction area and the melting or consolidation of the material correspondingly occurs in the respective layer in the form of “weld tracks” or “weld beads” so that finally, a plurality of layers formed from such weld tracks are present in the component. Components with a very high quality and breaking strength can now be produced in this manner.

However, it should be noted that the oxygen content in the processing chamber has an influence on the quality of the components. In particular, there is a correlation between the porosity of metallic components and the concentration of oxygen in the processing chamber. For this reason, for highest quality and strength components, the oxygen concentration during the build process in the processing chamber should be measured and should not exceed 1000 ppm, for example.

Even when, in practice, operations are carried out in a protective gas atmosphere in the processing area, as a rule, however, this is always “contaminated” by oxygen. This is particularly due to the fact that oxygen can permeate into the system through leaks. Even when the processing area itself is under a slight over-pressure, there are still points in the overall system, in the lines or the filters, at which an under-pressure may prevail. At these points, oxygen, for example, can permeate into the system and also into the processing chamber because of the movement of gas. However, oxygen can also be generated as moisture (water vapour) in the system. As a rule, the build material contains a certain residual moisture. This gets into the processing area by evaporation. In the region of the energy beam, water molecules can be split into oxygen atoms and hydrogen atoms due to its high energy and power; these then recombine in the form of molecular hydrogen and oxygen. Because of the relatively low concentrations of atomic hydrogen and atomic oxygen which are generated from the laser-induced splitting of water, it is also conceivable that the atomic hydrogen and atomic oxygen might not recombine fully and for this reason, atomic species could also be present in the processing chamber. In particular, atomic hydrogen may have a negative influence on the stability of a measurement of the oxygen concentration, as will be explained below.

The oxygen concentration (from molecular oxygen) in the processing area is measured with an oxygen sensor. In this regard, there are a variety of functional principles. Amperometric sensors and potentiometric sensors may be mentioned here in particular. In potentiometric sensors, a voltage or a resistance reflects the oxygen concentration. In amperometric sensors, oxygen molecules are ionised at a cathode and recombine at an anode, whereupon a current is produced which is proportional to the oxygen concentration.

The measurement of oxygen may, however, be erroneous under specific circumstances; this is in particular due to a cross-sensitivity of the sensor between hydrogen and water molecules. At low oxygen concentrations, these substances can produce erroneous signals in the sensor. These erroneous signals are primarily due to water and hydrogen molecules, which could potentially falsify the signal by various mechanisms or chemical reactions. Molecular hydrogen can react with molecular oxygen in the environment of the sensor, which generates water. This reaction can be caused by the increased operating temperature of the sensor. The chemical reaction between hydrogen and oxygen leads to a reduction in oxygen in the environment of the sensor. It follows from this that a low oxygen concentration is measured, but this no longer corresponds to the actual oxygen content in the processing chamber. In addition, water and hydrogen produce erroneous signals at low concentrations, as mentioned above. The reduction in the oxygen content in the environment of the sensor therefore leads to the occurrence of erroneous signals. As was stated, these erroneous signals are primarily due to water and hydrogen, but several factors contribute to these and the observed instabilities in the signal cannot be fully explained. It was observed that hydrogen has a tendency to be linked to an underestimate of the actual oxygen content, while water leads to an overestimation of the oxygen content. Both hydrogen as well as water can be adsorbed by the electrodes of the sensor, depending on the material of construction, and can respectively be split there into hydrogen anions and cations and hydroxide ions and hydrogen anions. The adsorbed state of molecular hydrogen and the corresponding splitting into hydrogen anions and cations can have a positive or negative influence on the electrical resistance (and correspondingly the electrical signal generated therefrom) of the electrodes, depending on the temperature and pressure conditions. The same effect may also occur with the absorption of atomic hydrogen, which may be generated in the processing chamber from the splitting of water, as explained above. Instabilities in the signal can therefore be explained by the adsorption of (atomic or molecular) hydrogen on the electrode surface. Furthermore, hydroxide ions can also be oxidized at an electrode in a similar manner to oxygen ions and hydrogen cations can be reduced, which in turn leads to an atmosphere which is enriched in molecular hydrogen and molecular oxygen. At a typical operating temperature for the sensor, water is regenerated from an atmosphere enriched in molecular hydrogen and oxygen. Thus, overall, the observed instabilities in the signal can also be explained by a chemical weighting between water, hydrogen and oxygen which is generated under the temperature conditions in the environment of the sensor and by the catalytic action of the sensor electrodes in the environment of the sensor.

In particular, the working principle of sensors which are based on the amperometric principle means that the signals behave in an unstable manner due to cross-sensitivity when the oxygen concentration drops to low values which are below a specified measurement threshold of the sensor (for example approximately <400 ppm), for example because of an increase in moisture or hydrogen at the sensor. The value of 400 ppm which is given by way of example here is not the desired concentration of oxygen in the processing area (which is approximately 0.1%, for example), but the threshold below which the measurement signal at the sensor typically becomes unstable. In particular, at a very low oxygen concentration (of <400 ppm, for example), a mutual generation of water and hydrogen/oxygen and therefore a severely fluctuating signal characteristic may be produced.

An objective of the present invention is to provide a sensor arrangement or an apparatus which exhibits stable signal behaviour even at low oxygen concentrations, so that an additive manufacture of a component can always take place under defined conditions.

This objective is achieved by means of a sensor arrangement in accordance with patent claim 1, an apparatus in accordance with patent claim 6 and a measurement method in accordance with patent claim 8.

A sensor arrangement in accordance with the invention serves in an apparatus for the additive manufacture of a component in a manufacturing process (“manufacturing apparatus”) in which build material, preferably comprising a metal powder, is consolidated on a construction area in a processing area by means of irradiation of the build material with at least one energy beam. The sensor arrangement comprises the following components:
a sensor module which is configured to detect oxygen molecules in a gas sample permeating into the sensor module and to generate an electrical sensor signal based on the quantity of the oxygen molecules,
a control module which is configured, by means of a comparison of the sensor signal or a variable derived from the sensor signal with a specified threshold value, to determine whether the sensor module is measuring outside a predetermined action range and if this is the case, to generate a control signal which is configured to initiate a predetermined countermeasure which is intended to modify the conditions in the apparatus in a manner such that the sensor module is again measuring in the action range.

A suitable sensor module is known in the prior art. The invention may be used in a particularly advantageous manner for amperometric or potentiometric sensors, but is also advantageous for other sensors. The sensor modules may be equipped with a reference sensor and/or a reference component which is capable of carrying out a reference measurement. In particular in the case of potentiometric sensors, a reference chamber or the reference volume is integrated into the sensor, wherein the reference volume is in direct contact with an electrode. The term “reference sensor” as used below should also be understood to mean the “reference component” of a sensor. In the case of amperometric sensors, the reference sensor and/or the reference component is an electrochemical reference cell, for example a solid cell (for example, solid cells produced from palladium, rhodium, rubidium and the corresponding oxides are used for the measurement), and in the case of potentiometric sensors, a reference gas volume is used.

It should be noted that a sensor generates its sensor signal as a function of the oxygen molecules present in the measuring zone. Thus, the sensor signal is first of all dependent on the quantity of oxygen molecules. However, because the measurement parameters are usually known (for example the volume of the gas sample and its pressure), usually, the oxygen concentration can be deduced directly from the sensor signal, or the sensor signal is a direct measure of the oxygen concentration. Preferably, the sensor signal is proportional to the oxygen concentration.

The sensor signal may be an analogue signal, in particular a voltage or a current, or a digital signal, for example a digital numerical value. Finally, an analogue signal may be obtained by conversion using an analogue-to-digital converter (ADC).

It should be noted here that, in particular in the case of amperometric sensors, water and hydrogen lead to interfering fractions in the sensor signal. As an example, in the case of an amperometric Nernst cell, a voltage is applied to the two platinum electrodes. At the cathode, which is in direct contact with the gas sample to be measured, oxygen molecules are reduced to oxygen ions (O²⁻). The oxygen ions diffuse through a solid electrolyte, for example a ZrO₂ plate, and are oxidized at the anode. The current generated by this oxidation is measured at the amperometer. This necessitates a reference measurement, which is carried using a Pd/PdO solid cell, for example. The effect described here also arises with a potentiometric sensor.

However, not only is oxygen present in the processing area-hydrogen and water are as well. Water is primarily generated from the moisture which is unavoidably contained in the powder. Some metal powders could also contain hydrogen, which can escape during the build process. Oxygen gets into the processing area primarily from points of the processing chamber which are not sealed.

In a first step, water (which escapes from the powder) is split into hydrogen atoms and oxygen atoms by the radiation in the energy beam (for example a laser beam); they recombine to generate molecular oxygen and molecular hydrogen. Hydrogen and oxygen can diffuse in the chamber to the sensor; it should be noted here that oxygen is taken up more readily by metal condensates. Thus, it can be assumed that oxygen and hydrogen are not in the chamber or at the sensor in a ratio of 1:2. As stated above, the oxygen present in the gas sample in the sensor originates primarily from the locations in the processing area which are not sealed and is not in a chemical relationship with the hydrogen, which is primarily generated from the splitting of water, or escapes directly from the metal powder.

However, water can also be generated from the reaction between oxygen and hydrogen at the sensor. This reaction is caused by the high temperature at the sensor (the operating temperature should be between 300-700° C. in order to enable the oxygen ions to diffuse through the ZrO₂ plate). This reaction leads to a reduction in the measured current at the sensor, because oxygen is consumed in this reaction and therefore no longer reaches the cathode.

In addition to oxygen, hydrogen can be split into hydrogen ions at the sensor and can lead to a current between the electrodes. When the oxygen concentration is significantly higher than the hydrogen concentration, oxygen can be reliably measured by the sensor. In the opposite case (excess of H₂), the hydrogen causes an instability in the sensor. Hydrogen can be adsorbed by platinum in two different states. In one of these states, the absorption of hydrogen brings about an increase in the electrical resistance of platinum; in the other, a reduction in the electrical resistance. The state of the adsorbed hydrogen depends in the first place on the temperature. Because of the high operating temperature of the sensor, hydrogen can change from one state to another and lead to instabilities in the signal.

Furthermore, water (H₂O) can be split into hydrogen anions (H⁺) and hydroxide ions (OH⁻) in the sensor. When at least a minimum concentration of water is present at the sensor (from the build material or from the reaction between hydrogen and oxygen), the water is adsorbed by the platinum. The ions which are generated behave in an analogous manner to oxygen at the sensor: hydrogen anions (H⁺) are reduced at the cathode and hydrogen diffuses into the chamber, while hydroxide ions (OH⁻) are oxidized at the anode in a similar manner to oxygen ions. Because of the oxidation of hydroxide ions, a current is measured at the amperometer as if oxygen were present in the chamber. This reaction profile is equivalent to the electrolysis of water. The concentration of oxygen is overestimated in this case. Water is consumed in this reaction, and in addition, hydrogen is generated in the chamber, which in turn leads to an unstable signal because of the absorption of the hydrogen onto the platinum or in turn to a consumption of oxygen (by the reaction between oxygen and hydrogen), and therefore to a reduction in the signal at the sensor or to an underestimation of the oxygen concentration.

The hydroxide ions may also lead to a current between the electrodes, which would likewise normally be far below the oxygen signal at low oxygen concentrations and high water concentrations, but may also be dominant.

As with any sensor, the sensor module has a measurement range in which it can carry out the measurements. This measurement range can be divided into two ranges: a stable range (in which the “action range” lies), in which oxygen can be correctly measured, and an unstable range, in which the aforementioned mechanisms distort the sensor signal. In this regard, the selection of the action range lies in the hands of the operator (however, it is always outside the unstable range, in the stable range). In the unstable range, the sensor signal usually rises again when the oxygen concentration is still falling after a certain turning point (in the case of an inverted sensor signal, this would be exactly the other way round). This turning point may be considered to be a possible threshold between the action range and an unwanted range (the rest of the measurement range). In this regard, it should be noted that the action range can in principle be freely selected (as long as it lies outside the unstable range) and therefore the unwanted range can as well. Beyond the turning point, the sensor signal is no longer monotonically related to the oxygen concentration, but is dominated by other effects (which is the reason for the increase).

In this regard, compared with the unwanted range, the action range is in principle the range with the higher oxygen concentration and always lies in the stable range of the sensor. An operator is not bound to use the turning point, but may also determine that beyond a specific oxygen concentration which is higher than at the aforementioned turning point, the action range has already been left (which is preferable). The action range then extends only to this point set by the operator. The action range may also be set from the first, for example to a fraction of the minimum desired oxygen concentration during the manufacture, for example 90% of the minimum concentration.

Within the action range, the sensor signal can be associated with a distinct oxygen concentration. The distinct correlation between the oxygen concentration and the sensor signal can also constitute a basis for the predetermination of the action range (the action range is the range with a higher oxygen concentration, in which the sensor signal has a specific relationship to the oxygen concentration, and preferably, the sensor signal is proportional to the oxygen concentration). The threshold between the action range and the unwanted range is characterized by a threshold value for the sensor signal. In this regard, it should be noted that after falling in the action range (when the oxygen concentration is falling), the sensor signal increases again in the unwanted range, which suggests a higher oxygen concentration. The threshold value characterizes the threshold between the action range and the unwanted range at which it specifies a value which the sensor module would display at this threshold, or the value for a variable which could be derived from the sensor signal, for example for an increase in the sensor signal with time. In the aforementioned example in which the point beyond which the sensor signal increases again for a falling oxygen concentration is selected as the threshold, the threshold value is the minimum of the sensor signal. In the other cited example, the threshold value is set by an operator (but should be above this minimum) and represents the threshold of the action range. As can be seen here, the action range may also be predetermined by setting the threshold value.

It should be noted here that when the threshold value corresponds to the minimum of the sensor signal, when the threshold value has been passed, measurements are already being made in the unstable range (unwanted range=unstable range). However, when in the unstable range, effective countermeasures are difficult to take because the sensor signals can no longer be relied upon. This means that the minimum cannot be determined in advance, because it is a function of the concentration of hydrogen and the moisture. It is therefore preferable to select the threshold value so that a minimum threshold is determined for the oxygen concentration which lies below the oxygen concentration specified for the build process (for example 0.06% at a desired concentration of 0.1%). Preferably, a threshold is less than 95% of the desired upper limit for the oxygen concentration for manufacture, particularly preferably less than 80%, in particular less than 50% or in fact less than 20%. Preferably, however, a threshold is set higher than the minimum threshold at which the sensor leaves its stable range. Now, what is determined (by calibration measurements or by calculations) is how high the sensor signal would be at this threshold. This sensor signal would now correspond to the threshold value.

In summary, it can be stated that the action range represents a range with a specific oxygen concentration in which the sensor module still delivers distinct measured values and the threshold value corresponds to a sensor signal (or a variable which can be derived from the sensor signal) at the threshold of the action range.

The control module now compares the sensor signal with the threshold value. As an alternative or in addition, the control module compares a variable derived from the sensor signal, for example its gradient, with the threshold value. This comparison determines whether the sensor module is measuring outside the action range. In the case of a sensor signal which falls as the oxygen concentration falls, this is, for example, outside the action range if it is below the threshold value. If it is reversed and increases as the oxygen concentration falls, then it is, for example, outside the action range when it is above the threshold value. In general, it can be stated that the unwanted range is when the sensor signal is beyond the threshold value.

If this is now the case, the control module generates a control signal which is configured to initiate a predetermined countermeasure which in turn is determined so as to modify the conditions in the apparatus in a manner such that the sensor module is again measuring in the action range.

It should be noted that the equipment for initiating countermeasures is firstly not an obligatory part of the sensor arrangement. In its simplest embodiment, the sensor arrangement only detects whether the measurements are being carried out in the unwanted region (i.e. outside the action range). However, components for initiating countermeasures may of course be present, for example a voltage controller or a temperature controller for the sensor module, which then can appropriately modify the voltage or the temperature by means of the sensor signal.

In the simplest case, the sensor signal may be a voltage (or also a logical zero), which is then appropriately registered by a manufacturing apparatus. As an example, the sensor signal is always at a logical “HIGH” level when measuring in the action range and falls to a logical “LOW” upon leaving the action range. By means of the “LOW”, the manufacturing apparatus detects that it has to initiate countermeasures. Clearly, the sensor signal may also be used the other way round (from “LOW” to “HIGH”) or comprise even more information, for example how deep into the unwanted range, or how severe the countermeasures should be.

A plurality of possible countermeasures exist which could be specified to modify the conditions in the apparatus in a manner such that the sensor module is again measuring in the action range. This might be the introduction of oxygen into the apparatus, for example. Because in normal sensors, the action range is only left far below the oxygen concentration used for a manufacturing process (for example at 400 ppm for a threshold of 1000 ppm for the manufacture), addition of a small quantity of oxygen would not exceed a threshold which was given for the manufacture, but would create an oxygen concentration in which the sensor module was once again measuring in the action range. However, an inert gas flow could also be increased as the countermeasure. The light gaseous hydrogen could collect in a sensor disposed in the top of the processing area. The increased flow of inert gas would cause stirring of the gas in the processing area and once again homogenize the atmosphere at the sensor module. This “blast cleaning” could bring the sensor module back to measuring in the action range.

An important aspect of the invention, however, is that the selection of the threshold value can produce a predicative initiation of countermeasures. Until now, in manufacturing procedures, a phenomenon occasionally occurs whereby a specific increase in the inert gas flow generates fluctuations which sometimes become stronger. The sensor module-inert gas pump system here leads to a build-up of the sensor signal. This often occurs when the inert gas pump triggers upon an increase in the sensor signal. An increase in the signal occurs when the oxygen concentration increases, but also when the sensor module starts measuring in the unstable range. If the inert gas flow is increased during the latter, it may have the effect that the oxygen concentration is further reduced. Although the sensor signal drops rapidly because of the inflow, after the inert gas flow is switched off, it immediately increases just as rapidly (because the sensor module now measures even “deeper” into the unstable range). After a short period, this in turn leads to the inert gas flow being switched on again, because the threshold is rapidly crossed again and a vicious circle is set up in which the sensor signal fluctuates severely. Note that an increase in the sensor signal above the threshold mentioned here implies that measurements have been carried out in the unstable range for some time (the minimum sensor signal “has been left way behind”), and therefore moved out of the action range some time ago.

This can be counteracted by the invention because here, moving out of the action range (which represents a part of the stable range) is the crucial factor for countermeasures. However, an advantageous comparison should take into account the fact that an increase in the sensor signal can in principle occur for three reasons (the two reasons discussed above can be divided into three reasons):
increase in oxygen concentration
reduction in oxygen concentration beyond a certain minimum concentration,
increase in concentration of moisture or hydrogen.

The last two points relate here to the unstable range; the last point relates to a shift in the threshold between the unstable range and action range.

The shift in the threshold will be discussed in more detail here. If the concentration of hydrogen or moisture (water vapour) increases, then the sensor module may be measuring in the unstable range even at higher oxygen concentrations, because effects of the unwanted gases dominate in the sensor module. This can be counteracted by a dynamic selection of the threshold value (minimum of the sensor signal) or by fixing the threshold value selection a little away from the minimum (a higher value than the minimum sensor signal). This ensures that even if the moisture and hydrogen concentrations fluctuate, the action range and the unstable range are always disjunctive.

The foregoing naturally applies to a sensor signal which falls with a falling oxygen concentration. It is the other way round for an inverted sensor signal (here, there is a maximum instead of a minimum).

When, as stated above, measuring starts in the action range and the sensor signal crosses the threshold value, then the unwanted range, but not necessarily the unstable range, is in fact entered. Because the most disadvantageous effects occur with measurements in the unstable range, with a suitable selection of the threshold value, a predicative initiation of countermeasures can be carried out. If the unwanted range is larger than the unstable range (threshold value always remote from the minimum of the sensor signal at higher oxygen concentrations), then countermeasures can already have been initiated by the control module before the unstable range is reached. An example would be the initiation of countermeasures when the sensor signal goes below the threshold value (in the case of a sensor signal which follows the oxygen concentration). In the case of an inverted sensor signal, it would exceed the threshold value.

An apparatus in accordance with the invention (“manufacturing apparatus”) serves for the additive manufacture of a component in a manufacturing process in which build material, preferably comprising a metal powder, on a construction area in a processing area is consolidated by means of irradiation of the build material with at least one energy beam. The apparatus comprises the following components:
a supply device for applying layers of build material to the construction area,
an irradiation device in order to selectively consolidate build material between the application of two layers of material by irradiation with at least one energy beam, as well as
a sensor arrangement in accordance with the invention.

The supply device (for example an arrangement for the layer by layer application of a metal powder) and the irradiation device (for example a laser) are known in the art. The characterizing feature is the sensor arrangement in accordance with the invention, which enables the oxygen concentration to be measured better. In addition to these components, the manufacturing apparatus may have further components, as are usually employed for manufacturing.

It should be pointed out at this juncture that the apparatus in accordance with the invention may also have a plurality of irradiation devices which can be controlled in an appropriately coordinated manner using control data. Only for the sake of completeness is it mentioned here that the energy beam may be both a particle beam as well as an electromagnetic beam such as a beam of light or, as is preferable, a laser beam.

A measurement method in accordance with the invention with a sensor arrangement in accordance with the invention in an apparatus in accordance with the invention comprises the following steps:
generating a sensor signal by means of the sensor module of the sensor arrangement,
comparing the sensor signal or a variable derived from the sensor signal with a specified threshold value and determining whether the sensor module is measuring outside a predetermined action range,
if the comparison reveals that the sensor module is measuring outside a predetermined action range: initiating a predetermined countermeasure which is intended to modify the conditions in the apparatus in a manner such that the sensor module is again measuring in the action range, wherein preferably, a control signal is produced with which the countermeasure can be initiated.

The measurement method (as well as the sensor arrangement) can operate in multiple stages. This means that a plurality of action ranges with a plurality of threshold values can be defined and when one of these threshold values is passed, a countermeasure associated with the respective threshold can be initiated.

An example of a countermeasure is a gas flow produced, for example, by means of a circulating pump or a pump for blowing in a protective or inert gas. In this regard, it does not necessarily have to be blown directly onto the sensor module. It is sufficient for the gas to move by the sensor, which can be brought about by a circulation in the processing area or by means of a flow of air through a line in which the sensor module lies.

Further particularly advantageous embodiments and developments of the invention will become apparent from the dependent claims as well as from the description below, wherein the independent claims of a category of claims can also analogously apply to the dependent claims and exemplary embodiments of another category of claims and in particular, individual features of different exemplary embodiments or variations may be combined to form novel exemplary embodiments or variations.

In accordance with a preferred sensor arrangement, the control module is configured to determine
whether the sensor signal lies over or under a threshold value which characterizes the threshold of the action range,
whether the variable derived from the sensor signal, preferably the first derivative with respect to time, lies over or under the threshold value,
whether a maximum or minimum of the sensor signal as the threshold value has been passed (this is with reference to the aforementioned turning point),
whether the sensor signal lies under or over a first threshold value and the variable derived from the sensor signal, preferably the first derivative with respect to time, lies under or over a second threshold value.

This is also the case for a preferred measurement method.

It should be noted here that in this respect, the conditions “under” and “over” always refer to whether the threshold value has been passed. In the case of a sensor signal which follows the oxygen concentration (falling sensor signal when the oxygen concentration falls), what is determined is whether it has dropped below the threshold value. In the case of a sensor signal which follows the inverse of the oxygen concentration (rising sensor signal when the oxygen concentration falls), what is determined is whether the threshold value has been passed. The term “derived variables” is a function of a comparison of the signal profile. For the most part, in the unstable range, larger increases are observed for smaller changes in concentration. On the other hand, preferably, it is precisely the unstable range that is avoided by the countermeasures. However, it may be the case that the gradient of the sensor signal increases or decreases for a uniform reduction in the oxygen concentration even before the turning point. This effect could be used for the comparison.

The comparison is preferably additionally carried out on the basis of a plurality of further measured values, in particular with a plurality of measured values from the group measuring the moisture content, a further sensor signal, the measured temperature and the measured pressure. If, for example, the moisture content is known, then the turning point or a dynamic threshold value can be calculated. By a comparison with the original sensor signal, another sensor signal could provide an indication as to whether it is still in the action range, or as to how far it still is from the threshold value. The temperature and pressure may also be helpful. It should be noted here that only the sensor signal is present and its values in the unstable range very strongly resemble values in the action range.

In accordance with a preferred sensor arrangement, the sensor signal is smoothed mathematically or by means of a smoothing unit. This has the advantage that smaller fluctuations can still have no effects on the initiation of countermeasures. In the analogous circuit technology, this is also described as “debouncing” In this regard, in addition to the mathematical method described below, circuits for smoothing may also be used, for example an integration of a capacitor current or a conversion of the signal into a digital number within a time window. In this regard, preferably, a sliding time window is used in which the value for the oldest sensor signal is eliminated when a new sensor signal arrives.

For the purposes of smoothing, the control module preferably comprises a timer and the comparison of the sensor signal or a variable derived from the sensor signal with the specified threshold value is based on one or more of the possibilities listed below:

Determining whether the comparison has the same result within a predetermined smoothing period. As an example, whether the sensor signal is below the threshold value over the smoothing period is determined.

Integrating the sensor signal over a specified integration period and comparing the integral with the threshold value.

Forming a statistical mean over a plurality of sensor signals within a specified measuring period.

The periods here are preferably longer than 0.1 s, in particular longer than 1 s. However, the periods are preferably shorter than 60 s, in particular shorter than 30 s.

A preferred sensor arrangement comprises a selective filter element which can be used as the countermeasure for filtering the gas sample. In this regard, the filter element is configured to filter a gas sample (P) so that at least hydrogen molecules and/or water molecules and/or hydroxide ions and/or hydrogen ions are filtered out of the gas sample (P), wherein the filter element is preferably formed by an adsorbent, a molecular sieve or by an electrode of the sensor module, wherein in particular, the electrode material of the electrode is selected such that the conversion of water vapour to hydrogen ions and hydroxide ions and/or the conversion of water to hydrogen and oxygen and/or the generation of water from hydrogen and oxygen and/or the adsorption of water vapour is inhibited (or even completely prevented).

In accordance with a preferred sensor arrangement, the sensor module has (at least) an anode and (at least) a cathode and in particular is an amperometric or potentiometric sensor. In this regard, preferably, the sensor arrangement comprises a solid electrolyte between the anode and cathode, in particular zirconium dioxide (ZrO₂).

A preferred sensor arrangement comprises a controller which is configured to control the operating voltage and/or operating temperature of the sensor module. In this regard, the voltage or temperature is kept within a predetermined range in which an ionisation, accumulation or deposition of water vapour and/or hydrogen is inhibited or even (completely) prevented. A voltage here is preferably decreased below a predetermined threshold value. Preferably in this regard, the operating voltage of the sensor is decreased by ±0.8/0.9V to ±0.3/0.4V. It is generally preferable to operate the sensor at a temperature between 300-700° C.

A preferred sensor arrangement comprises a reference sensor module (wherein this term may also mean a sensor with a reference component) which is preferably operated at a different voltage than the sensor module, in particular at a lower voltage. As an alternative or in addition, the reference sensor module comprises a reference gas in a reference chamber as the gas sample (preferably in the case of a potentiometric sensor module). As an alternative or in addition, the reference sensor module comprises a reference measurement cell, preferably a solid cell, for example produced from palladium or palladium oxide. This embodiment preferably concerns an amperometric sensor module.

A preferred apparatus comprises components which can be used in order to initiate countermeasures, wherein the control module of the sensor arrangement is connected to the apparatus for the purposes of signalling, in particular electronically, in a manner such that these countermeasures can be initiated with components of the apparatus by means of the control signal of the control module.

A preferred component for initiating a countermeasure is a gas flow device (for example a valve controller or a gas pump), configured for the production of a flow of gas in the processing area. This should be understood to mean an element with which a pressure can be applied to a gas or with which a gas can be moved. In this regard, the apparatus is configured in a manner such that a gas sample can be moved by means of the gas sample to the sensor arrangement, preferably wherein the gas sample is configured in a manner such that the volume of gas in a processing area of the apparatus is circulated, a volume of gas is discharged from the processing area or a gas, in particular an inert gas, is introduced into the processing area.

The gas flow device may, however and preferably, comprise an inlet valve and an outlet valve, wherein the control signal is configured in a manner such that the inlet valve and the outlet valve are open. In this manner, gas and therefore also moisture and hydrogen are discharged from the processing area. Preferably in this regard, the apparatus comprises a gas channel and the sensor module of the sensor arrangement is disposed in this gas channel in a manner such that gas flowing through the gas channel serves as the gas sample for a measurement. Preferably, gas is discharged from the processing area through the gas channel in which the sensor module of the sensor arrangement is placed. Optionally, an outlet valve is disposed between this gas channel and the processing area.

In a preferred measurement method, the threshold value and the predetermined action range are (automatically) determined and whether the sensor module is measuring within the action range is checked. This may, for example, be carried out by measuring the moisture content or by a test run with a known modified oxygen concentration.

In a preferred measurement method, a measuring curve is generated from a plurality of sensor signals at different times and a local maximum or minimum of the measuring curve is determined and this is used as the measure regarding whether the sensor signal lies beyond the threshold value.

In a preferred measurement method, a maximum or minimum value for a standard deviation of the sensor signal is specified as the threshold value.

In a preferred measurement method, the manufacturing process which was selected is checked and the threshold value and therefore the action range is determined as a function of this manufacturing process, in particular based on a total irradiation period per layer and/or a quantity of the build material which is used. In addition, a countermeasure may be automatically selected from a specified number of countermeasures as a function of the manufacturing process.

In a preferred measurement method, for a reference measurement, the partial pressure of oxygen in the sensor module is reduced to a specified minimum concentration. This is preferably carried out by pumping with a voltage or by an oxygen adsorbent or by specific flushing of the sample gas. Pumping with a voltage may be carried out by applying a voltage to the (platinum) electrodes of the sensor module which, for example, may be higher than the operating voltage for the measurement of the oxygen concentration, whereupon this functions as an oxygen pump (by ionisation and motion in an electrical field). In this regard, oxygen is guided from the atmosphere to be measured through the electrolytes. As an alternative or in addition, the voltage applied to the electrodes may be reversed compared with the operating voltage, so that the oxygen content in the reference sensor module is reduced.

In a preferred measurement method, a reference measurement is carried out with the sensor module at other preadjusted settings than for the generation of the sensor signal. Preferably, this is carried out at another operating voltage, wherein the other preadjusted settings are selected in a manner such that a signal which is based on hydrogen and/or water vapour is suppressed and a ratio of oxygen to hydrogen and/or water vapour is determined on the basis of a reference signal obtained from this reference measurement and the sensor signal. In this regard, it is preferable for a correction of the sensor signal to be carried out on the basis of the determined ratio.

In a preferred measurement method, a specific countermeasure is selected or a plurality of countermeasures are selected. Depending on the sensor signal or a modification to the sensor signal, in this regard, different countermeasures may also be selected for different modifications. A group of countermeasures is listed below, from which one or more measures may be selected as simultaneously used or alternative (or sole) countermeasures:
Increasing the power of a pump of the apparatus, in particular a pump for letting in a protective gas or inert gas. This countermeasure has already been described above and serves for the homogenization of the atmosphere inside the processing area.
Opening a valve of the apparatus, preferably an inlet valve and/or an outlet valve. This countermeasure has been described above and serves to change the atmosphere inside the processing area.
Simulating a sensor signal, in particular with a predicative component. This will be described below.
Introducing oxygen into the processing area. This step, which at first sight appears counter-intuitive, is based on the fact that in the unstable range, a significantly smaller oxygen concentration is present than the desired upper limit for the oxygen concentration during the manufacture (for example, 400 ppm instead of the upper limit of 1000 ppm). Deliberately allowing oxygen to enter at least up to the upper limit is therefore entirely possible. Exactly as much oxygen can be let in for the threshold value to be crossed again, i.e. measurement is again taking place in the action range. The upper limit depends strongly on the type of manufacture and the material used. As an example, in some processes, instead of 1000 ppm (0.1%), an upper limit of only 500 ppm (0.05%) is desired, or even of 13000 ppm (1.3%). The minimum limit for the stable range of an oxygen sensor is, however, such that even values below the upper limit can be measured. According to this, as much oxygen is introduced into the processing area for the sensor to be measuring in the stable range, but the oxygen concentration lies below the upper limit specified for the respective manufacturing process.
Removing water vapour and/or hydrogen from the processing area. This may in particular be carried out using a filter unit which selectively adsorbs water vapour and/or hydrogen.
Reducing the sensitivity to water and/or the sensitivity to hydrogen of the sensor module. This may, for example, be carried out by changing the operating voltage.
Changing the sensor module for other measurements of a sensor signal. In this regard, another oxygen sensor is used as soon as the action range has been moved away from.
Using a second sensor, which in particular is disposed at another position, for the measurement of a further sensor signal, wherein the comparison is preferably additionally based on the further sensor signal, in particular on a signal ratio of the sensor signal to the further sensor signal. There are two preferred types of second sensor module. The first is an oxygen sensor which is simply coupled up when the first sensor module no longer measures reliably. The other one is a moisture sensor which can be used for a predicative algorithm.
Changing the manufacturing mode of the apparatus, in particular in the form of an interruption. This may be undertaken until the action range is once more obtained.
Changing the detection mode of the sensor, in particular by applying another voltage or another temperature, in particular for a reference measurement. In addition, such a reference measurement with the sensor module at a different temperature and/or voltage and comparison of the behaviour of a reference curve obtained in that manner with a measuring curve produced from sensor signals may constitute a countermeasure, because in this manner, out of the action range, the countermeasures which will return it to the action range can be identified. If an estimate is obtained as to how much the sensor signal deviates from the actual oxygen concentration, other countermeasures can be specifically implemented.
Reversing the polarity of the electrodes of the sensor module. The reversal of the polarity can produce an “electrical pumping” of the sensor module. However, a reversal of the polarity also leads to a reversal of a reference/measurement sample.
Changing a mode for the control of the inflow of inert gas. In addition to the force, this may also be the direction or the composition of the gas.

When simulating the measuring curve of the sensor signal, the aim is to interpolate a future profile from the previous reliable measurement points as soon the action range is left, because from this point, the sensor signal can no longer be considered to be reliable (at least when the action range comprises the entire stable range of the sensor module). As an alternative or in addition, “prior knowledge” or a reference measurement for a simulation may be employed. For the measuring curve, then, based on the previous reliable measurement points and on measurement points from a previous measurement or from a reference measurement, its future profile can be simulated as soon as it has moved away from the action range. Furthermore, as an alternative or in addition, a predicative algorithm together with a coupling with a moisture sensor may be employed. In this case, it is assumed that hydrogen is generated from splitting water and then reacts at the sensor with oxygen which permeates through unsealed sites, whereupon finally, the chemical weighting and the corresponding instabilities in the signal are generated.

In accordance with a preferred measurement method or in a preferred sensor arrangement, the operation is multi-staged. In this regard, a plurality of action ranges are defined with a plurality of (different) threshold values and when one of these threshold values is passed, one of the respectively associated countermeasures is initiated. As an example, when a first threshold value is crossed, an airflow is strengthened and when a second threshold value is crossed, a filter unit is moved in front of the filter module and when a third threshold value is crossed, oxygen is blown into the processing area.

An advantage of the invention is that when an unstable range is reached for the sensor module, countermeasures can counteract them. In this unstable region, it is no longer possible to produce a reliable measuring state. In addition, it is also difficult to return to the action range of the sensor module because the sensor signals can no longer be relied upon and basically, the action range has to be returned to by “flying blind”.

The invention will now be described again in more detail with reference to the accompanying figures and with the aid of exemplary embodiments. In this regard, in the various figures, identical components are provided with the same reference symbols. In the figures:

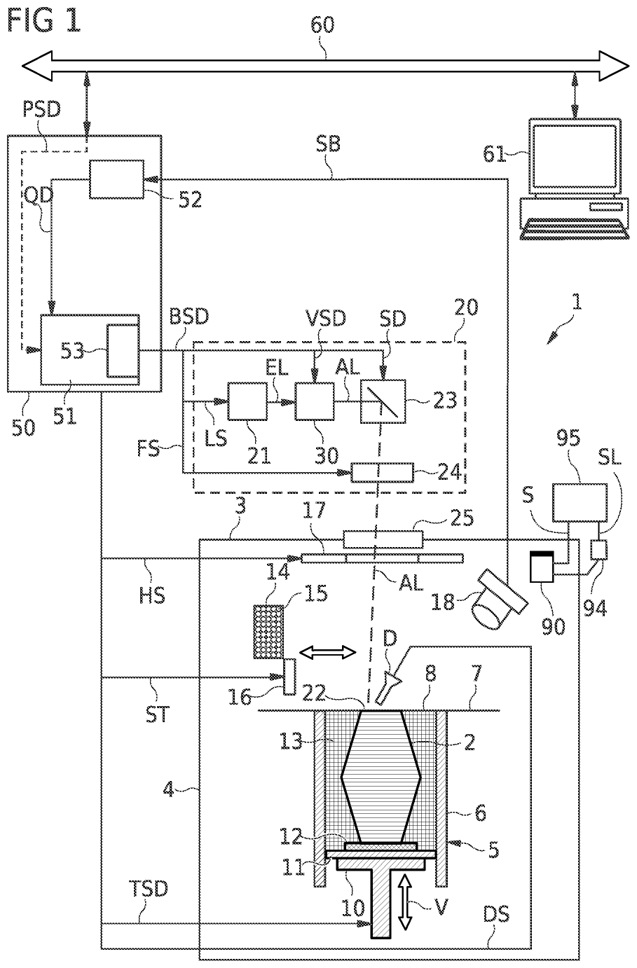

FIG. 1 shows a diagrammatic, partially sectional view of an exemplary embodiment of an apparatus for additive manufacture with a sensor arrangement in accordance with the invention,

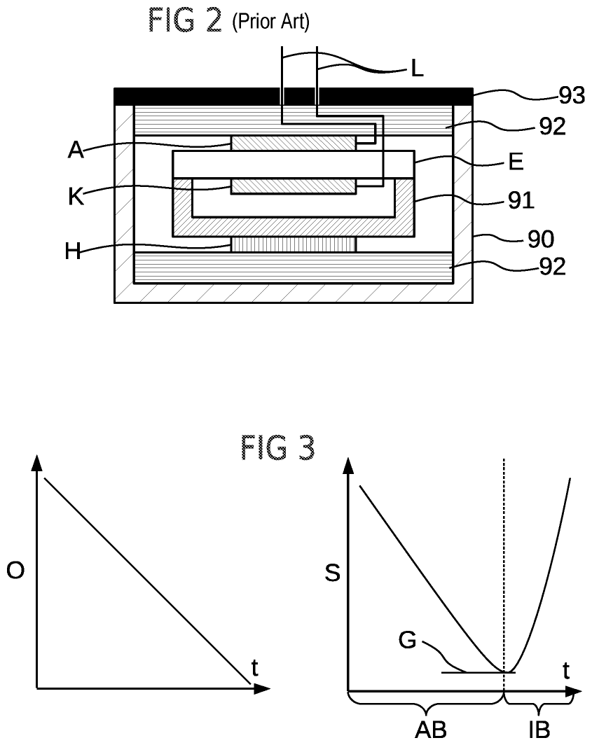

FIG. 2 shows a sensor module in accordance with the prior art,

FIG. 3 shows an example of a possible profile for a sensor signal when the oxygen concentration falls,

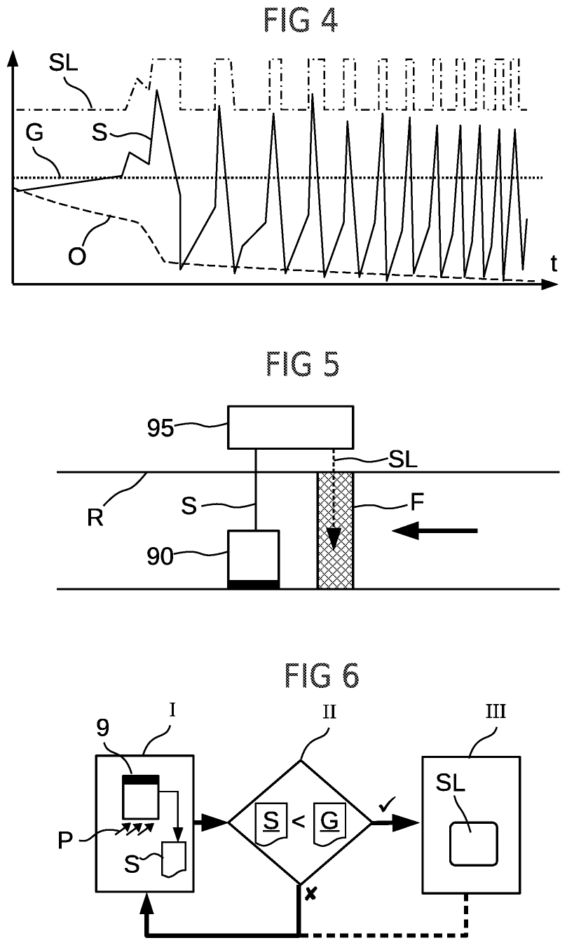

FIG. 4 shows unstable measurement behaviour,

FIG. 5 shows an example of a sensor arrangement in accordance with the invention with filtration as the countermeasure.

FIG. 6 shows a block diagram of possible steps of the method in a preferred measurement method.

The exemplary embodiments below are described with reference to an apparatus 1 for the additive manufacture of components in the form of a laser sintering or laser melting apparatus 1, wherein it is explicitly stated once again that the invention is not limited to laser sintering or laser melting apparatuses. The designation of the apparatus is therefore abbreviated below to a “laser sintering apparatus” 1 without any limitation to the generality.

A laser sintering apparatus 1 is shown diagrammatically in FIG. 1. The apparatus has a processing chamber 3 or a processing area 3 with a chamber wall 4 in which the manufacturing process is substantially carried out. In the processing chamber 3 is a container 5 which is open to the top and which has a container wall 6. The upper opening of the container 5 forms the respectively current working plane 7. The region of this working plane 7 which lies inside the opening of the container 5 can be used for the construction of the object 2 and is therefore designated as the construction area 8.

The container 5 has a base plate 11 which can be moved in a vertical direction, which is disposed on a carrier 10. This base plate 11 closes the container 5 from below and therefore forms its bottom. The base plate 11 may be formed integrally with the carrier 10, but it may also be separate from the plate forming the carrier 10 and be attached to the carrier 10 or simply mounted on it. Depending on the specific type of the build material, i.e. the powder used and the manufacturing process, for example, a construction platform 12 may be attached to the base plate 11 as a construction substrate, on which the object 2 is constructed. In principle, however, the object 2 may also be constructed on the base plate 11 itself, which then forms the construction substrate.

The basic construction of the object 2 is carried out by initially applying a layer of build material 13 to the construction platform 12, then selectively consolidating the build material 13 with a laser beam AL as the energy beam at points which are to form parts of the object 2 to be manufactured, then the base plate 11, and therefore the construction platform 12, is lowered with the aid of the carrier 10 and a new layer of the build material 13 is applied and selectively consolidated, and so on. FIG. 1 shows the object 2 constructed in the container in an intermediate state on the construction platform 12 below the working plane 7. It already has a plurality of consolidated layers, surrounded by build material 13 which has remained non-consolidated. A variety of materials may be used as the build material 13, preferably powders, in particular metal powders, plastic powders, ceramic powders, sand, filled or mixed powders, and also pasty materials. The invention is of particularly advantageous application to metallic build materials 13.

Fresh build material 15 is located in a reservoir 14 of the laser sintering apparatus 1. The build material can be applied to the working plane 7 or inside the construction area 8 in the form of a thin layer with the aid of a coater 16 which can be moved in a horizontal direction (double-headed arrow).

Optionally, an additional radiant heater 17 is located in the processing chamber 3. This may serve to heat the applied build material 13 so that the irradiation device used for the selective consolidation does not have to introduce too much energy. This means that, for example with the aid of the radiant heater 17, a basic quantity of energy has already been introduced into the build material 13, which naturally is still below the energy necessary for the build material 13 to melt or in fact sinter. An example of a radiant heater 17 is an infrared emitter.

For the selective consolidation, the laser sintering apparatus 1 has an irradiation device 20, or specifically an illumination device 20, with a laser 21. This laser 21 produces a laser beam EL which is initially supplied to a beam formation device 30 (as the input energy beam EL or input laser beam EL). As has already been described above, the beam formation device 30 may then be used in order to modify the intensity distribution, i.e. the profile of the intensity of the energy beam, in order to overlay a top hat profile on top of a Gaussian profile. To this end, the beam formation device 30 may be controlled with suitable intensity distribution control data, VSD.

A preferred beam formation device 30 here may firstly, for example on the inlet side, have a beam divider in the form of a thin layer polarizer which divides the incoming laser beam EL into two linearly polarised part-beams. Each of these linearly polarised part-beams can be guided to a separate beam formation element. These beam formation elements are responsible for the actual formation of the beam. In this regard, it may, for example, be what is known as a passive DOE (DOE=Diffractive Optical Element) which operates by reflection and modifies the wave front of the incident part-beam through local modulation of the phase and/or amplitude. An example of this is a LCoS Micro-Display (LCoS=Liquid Crystal on Silicon), which can be controlled with the appropriate intensity distribution control data, VSD, which can be delivered from the beam control interface 53 of the control device 50 of the laser sintering apparatus 1 which will be described below.

The (output) energy beam or laser beam AL, optionally modified by the beam formation device, is then deflected via a downstream deflection device 23 (scanner 23) in order thereby to traverse the consolidation pathways (i.e. the illumination pathways or tracks) provided in accordance with the illumination strategy in the respective layer to be consolidated and to selectively introduce the energy. This means that by means of the scanner 23, the impact surface 22 of the energy beam AL is moved on the construction area 8, whereupon the current movement vector or the direction of movement (scanning direction) of the impact surface 22 on the construction area 8 can be modified frequently and rapidly. In this regard, this laser beam AL is focussed through a focussing device 24 onto the working plane 7 in a suitable manner. The irradiation device 20 here is preferably located outside the processing area 3 and the laser beam AL is guided into the processing area 3 via a coupling window 25 in the chamber wall 4 attached to the top of the processing area 3.

The irradiation device 20 may comprise not just one but a plurality of lasers, for example. Advantageously in this regard, it may be a gas or solid state laser or any other type of laser, such as laser diodes, for example, in particular VCSEL (Vertical Cavity Surface Emitting Laser) or VECSEL (Vertical External Cavity Surface Emitting Laser), or a line of these lasers. More particularly preferably, in the context of the invention, one or more unpolarised single mode lasers, for example a 3 KW fibre laser with a wavelength of 1070 nm, may be employed.

In the exemplary embodiment shown, an optional, preferably displaceable and/or movable nozzle D is disposed in the processing chamber 3 and can be used to supply a gas or a mixture of gases into the region of the impact surface of the laser beam AL on the construction area 8 in order to influence the nominal weld depth in this manner.

Furthermore, the laser sintering apparatus 1 contains a detector assembly 18 which is suitable for detecting a process beam emitted during the impact of the laser beam AL on the build material in the working plane. This detector assembly 18 operates with spatial resolution, i.e. it is capable of detecting a kind of emission image of the respective layer. Preferably, an image sensor or a camera 18 which is sufficiently sensitive in the range of the emitted radiation is used as the detector assembly 18. As an alternative or in addition, one or more sensors for detecting an optical and/or thermal process beam could be used, for example photodiodes, which detect the electromagnetic radiation emitted from a meltpool under the impact of the laser beam AL, or temperature sensors for detecting an emitted thermal radiation (what is known as meltpool monitoring). An association of the signal from a non-spatially resolved sensor with the coordinates would be possible, wherein the coordinates which are used to control the laser beam are temporally associated with the respective sensor signal. In FIG. 1, the detector assembly 18 is shown inside the processing chamber 3. However, it could also be located outside the processing chamber 3 and the process beam would then be detected through a further window in the processing chamber 3.

The signals detected by the detector assembly 18 may be transferred here as a processing area sensor data set or slice image SB to a control device 50 of the laser sintering apparatus 1, which also serves to place the various components of the laser sintering apparatus 1 under the overall control of the additive manufacturing process.

To this end, the control device 50 comprises a control unit 51 which controls the components of the irradiation device 20 via an irradiation control interface 53, namely in this case sends laser control data LS to the laser 21, sends intensity distribution control data VSD to the beam formation device 30, sends scan control data SD to the deflection device 23 and sends focus control data FS to the focussing device 24. The totality of these data may be designated as illumination control data, BSD.

The control unit 51 also controls the radiant heater 17 by means of suitable heating control data HS, controls the coater 16 by means of coating control data ST and controls the movement of the carrier 10 by means of carrier control data TSD, and therefore controls the layer thickness. Furthermore, it also controls the nozzle D with the aid of nozzle control data DS.

In addition, the control device 50 here has a data quality determination device 52 which contains the processing area sensor data set SB and determines the data quality based on it which can, for example, be transmitted to the control unit 51 in order to be able to engage with the additive manufacturing process for control purposes.

The control device 50 is coupled to a terminal 61 with a display or the like, for example in this case via a bus 60 or another data connection. Via this terminal, an operator can control the control device 50 and therefore the entire laser sintering apparatus 1, for example by transmitting process control data PSD.

In this example, the apparatus 1 comprises a sensor arrangement 9 in accordance with the invention. A sensor module 90 is disposed in the upper right corner of the processing area 3 and measures the oxygen concentration in the processing area 3. The sensor arrangement 9 furthermore comprises a control module 95 which is configured, by means of a comparison of the sensor signal S or a variable derived from the sensor signal S with a specified threshold value G, to determine whether the sensor module 90 is measuring outside a predetermined action range AB (see FIG. 3) and if this is the case, to generate a control signal SL which is configured to initiate a predetermined countermeasure which in turn is set up to change the conditions in the apparatus 1 in a manner such that the sensor module 90 again measures in the action range AB (see the description of the measurement method in accordance with FIG. 6).

In this example, the sensor arrangement 9 also comprises a controller 94 which is configured to regulate the operating voltage and/or operating temperature of the sensor module 90 as a countermeasure so that there an ionisation, accumulation or deposition of water vapour and/or hydrogen is reduced. To this end, the controller 94 is in signalling communication with the control module 95, so that it receives a control signal SL from it and can initiate the countermeasures with this control signal SL.

FIG. 2 shows an example of a sensor module 90 in accordance with the prior art as can be used in a sensor arrangement 9 in accordance with the invention of FIG. 1. The outside of the sensor module 90 comprises a gas-permeable sensor housing 90 (which is shown here also with the components contained in the sensor module 90), which is covered by a cover 93. The sensor arrangement 9 can be attached to the chamber wall 4 of the processing chamber 3 by means of this cover 93. Lines L for the sensor module 90 can be fed out through a hole in the chamber wall 3. It is also possible, however, for the cover 93 to be somewhat longer and provided with a screw thread so that the sensor module can be passed from outside through a hole in the chamber wall 4 and can be screwed in there.

The functional components of the sensor module 90 are disposed inside the sensor housing 90. They are the anode A and the cathode K, which are separated by a solid electrolyte E, for example zirconium dioxide, and the heater H for heating the inner chamber 91, in which the cathode K is disposed. This functional construction is retained by two fixing elements 92, for example glass wool. The voltages or currents at the electrodes A, K are conveyed out by means of the lines L where they can be processed further. The heater H also has two lines for supplying energy, but are not shown for the sake of clarity.

FIG. 3 shows an example of a possible profile of a sensor signal S when the oxygen concentration falls. On the left is shown the oxygen concentration O during the measurement period t. It falls consistently. On the right is the sensor signal S within the measurement period t at this oxygen concentration O. As can be seen, in the action range AB, the sensor signal falls strictly monotonically with the falling oxygen concentration O, so that an exact association of sensor signal S and oxygen concentration O is possible. However, when the oxygen concentration O passes below a certain limit (the time is indicated as a dashed line), then the sensor moves into an unstable range in which the sensor signal S no longer constitutes a measure of the oxygen concentration O (here, the signal rises again). The minimum of the curve may be set as the threshold value G. However, in practice, the threshold value G is preferably set further up, so that the unstable range IB is not reached, because this can also fluctuate depending on the concentration of moisture and hydrogen.

FIG. 4 shows an unstable measurement behaviour which can occur in the unstable range and is highly disadvantageous. Here, it is almost impossible to move out of the unstable region IB again (see FIG. 3). The oxygen concentration O (dashed line), the sensor signal S (solid line) and a control signal SL (dot-dash line) are shown here. In the case shown here, which is not in accordance with the invention, just be crossing the threshold value G, the sensor signal S will be set off a trigger and the trigger switches off when the sensor signal S has dropped again below the threshold value G. The oxygen concentration shows, however, that it is already in the unstable range. According to FIG. 3, the increase in the fluctuations occurs simply because of the behaviour of the sensor in the unstable range. The fluctuations which become stronger and stronger can no longer be stopped by the underlying controller.

This arises because no check has been carried out as to whether the given threshold condition has been checked, or whether the action range AB (see FIG. 3) has been moved out of, and the initiated countermeasure is not suitable for a return to the action range.

FIG. 5 shows an example of a sensor arrangement 9 in accordance with the invention with a filter as the countermeasure. The sensor arrangement 9 is disposed in a conduit R (as the gas channel). The sensor module 90 shown in FIG. 2 may be considered to be the sensor module 90. The filter element F here is disposed in the conduit R and gas which flows through the conduit R (arrow) must first pass the filter element F before it meets the sensor module 90. In this manner, water is filtered out of the gas flow, which means that the unstable range is shifted to a lower oxygen concentration O, which in this example also means a shift of the threshold value G to a lower oxygen concentration O.

However, the filter module F is not in the position shown all the time. Only when it is confirmed that the sensor module 90 is no longer measuring in an action range AB (see FIG. 3) is a control signal SL generated by the control module 95 on the basis of the sensor signal S, which means that the filter element F is moved into the conduit R, as shown by the arrow in dashed lines.

A two-stage countermeasure may even be implemented. In a first stage, in which it moves below a first threshold value, the gas flow is simply increased (arrow in solid lines); in a second stage, which moves below a further threshold value, the filter element F is moved into the conduit R.

FIG. 6 shows a block diagram of a possible procedural profile for a preferred measurement method with a sensor arrangement 9 as shown in the preceding figures, for example, in an apparatus 1 as shown in FIG. 1, for example.

In step I, a gas sample P in the processing area 3 of the apparatus 1 reaches the sensor module and a sensor signal S is generated by the sensor module 90 of the sensor arrangement 9.

In step II, a sensor signal S is compared with a specified threshold value G and whether the sensor module 90 is measuring outside a predetermined action range AB is determined. In this example, this is the case when the sensor signal S is smaller than the threshold value G.

If the sensor signal S is larger than the threshold value G, then the measurement is repeated consistently.

In step III, the comparison has shown that the sensor module 90 is measuring outside a predetermined action range AB (sensor signal S is smaller than the threshold value G). In this case, a control signal SL is produced with which a predetermined countermeasure can be initiated, in this case, for example, an opening of ventilation valves. By means of the countermeasure, the conditions in the apparatus 1 are modified in a manner such that the sensor module 90 again measures in the action range AB.

The dashed lines indicate that afterwards, the measurement method is to be continued.

Finally, it should be indicated once again that the apparatuses described in detail above relate only to exemplary embodiments which could be modified by the person skilled in the art in very different manners without departing from the scope of the invention. Furthermore, the use of the indefinite article “a” or “an” does not exclude the fact that the features in question could also be present in a plurality thereof. Similarly, the term “unit” does not exclude the fact that this could consist of a plurality of cooperating secondary components which could optionally also be distributed in space.

### LIST OF REFERENCE SYMBOLS

1 apparatus for additive manufacture/laser sintering apparatus
2 component/object
3 processing area/processing chamber
4 chamber wall
5 container
6 container wall
7 working plane
8 construction area
9 sensor arrangement
10 carrier
11 base plate
12 build platform
13 build material (in container 5)
14 reservoir
15 build material (in reservoir 14)
16 coater
17 radiant heater
18 sensor arrangement/camera
20 irradiation device/illumination device
21 laser
22 impact surface (of laser beam)
23 deflection device/scanner
24 focussing device
25 coupling window
30 beam formation device
50 control device
51 control unit
52 data quality determination device
53 irradiation control interface
60 bus
61 terminal
90 sensor module/sensor housing
91 inner chamber
92 fixing element
93 cover
94 controller
95 control module
A anode
AB action range
AL (output) energy beam/laser beam
BSD control data/illumination control data
D nozzle
DS nozzle control data
E solid electrolyte
EL input energy beam/laser beam
F filter element
FS focussing control data
G threshold value
H heating
HS heating control data
IB unstable range
K cathode
LS laser control data
O oxygen concentration
P gas sample
PSD process control data
QD quality data
R conduit
S sensor signal
SB processing area sensor data set/slice image
SD scan control data
SI layer information
SL control signal
SM hatched segment/strips
ST coating control data
TSD carrier control data
VSD intensity distribution control data

1. A sensor arrangement for an apparatus for the additive manufacture of a component in a manufacturing process in which build material, is consolidated on a construction area in a processing area by means of irradiation of the build material with at least one energy beam, the sensor arrangement comprising:
a sensor module which is configured to detect oxygen molecules in a gas sample permeating into the sensor module and to generate an electrical sensor signal based on the quantity of the oxygen molecules,
a control module which is configured, by means of a comparison of the sensor signal or a variable derived from the sensor signal with a specified threshold value, to determine whether the sensor module is measuring outside a predetermined action range and if this is the case, to generate a control signal which is configured to initiate a predetermined countermeasure which is intended to modify the conditions in the apparatus in a manner such that the sensor module is again measuring in the action range.

2. The sensor arrangement of claim 1, wherein the control module is configured to determine:
whether the sensor signal lies over or under a threshold value which characterizes the threshold of the action range,
whether the variable derived from the sensor signal lies over or under the threshold value, and wherein the variable is the first derivative with respect to time,
whether a maximum or minimum of the sensor signal as the threshold value has been passed,
whether the sensor signal lies under or over a first threshold value and the variable derived from the sensor signal lies under or over a second threshold value,
wherein the comparison is additionally carried out on the basis of a plurality of other measured values, with a plurality of measured values from the group formed by measured moisture content, a further sensor signal, measured temperature and measured pressure.

3. The sensor arrangement of claim 1, wherein the sensor signal is smoothed mathematically or by means of a smoothing unit, wherein the control module comprises a timer, and the comparison of the sensor signal or a variable derived from the sensor signal with the specified threshold value
is carried out on the basis of whether this comparison has the same result within a predetermined smoothing period, or
is carried out on the basis of a sensor signal integrated over a specified integration period, or
is based on a statistical mean of the sensor signal which was obtained from a plurality of sensor signals over a specified measuring period, wherein a sliding time window is used.

4. The sensor arrangement of claim 1, comprising a selective filter element which can be used as a countermeasure for filtering the gas sample, the filter is configured to filter a gas sample so that at least hydrogen molecules and/or hydrogen ions and/or water molecules and/or hydroxide ions are filtered out of the gas sample, wherein the filter element is formed by an adsorbent, a molecular sieve or by an electrode of the sensor module, wherein the electrode material of the electrode is selected such that the conversion of water vapour to hydrogen ions and hydroxide ions and/or the conversion of water to hydrogen and oxygen and/or the generation of water from hydrogen and oxygen and/or the adsorption of water vapour and/or hydrogen is inhibited.

5. The sensor arrangement of claim 1, comprising a controller which is configured to control the operating voltage and/or operating temperature of the sensor module, in a manner such that an ionisation, accumulation or deposition of water vapour is prevented, wherein the voltage is dropped below a specified threshold value.

6. An apparatus for the additive manufacture of a component in a manufacturing process in which build material on a construction area in a processing area is consolidated by means of irradiation of the build material with at least one energy beam, the apparatus comprising:
a supply device for applying layers of build material to the construction area,
an irradiation device in order to selectively consolidate build material between the application of two layers of material by irradiation with at least one energy beam, as well as
a sensor arrangement as claimed in claim 1.

7. The apparatus of claim 6, comprising components which can be used in order to initiate countermeasures, wherein the control module of the sensor arrangement is connected to the apparatus for the purposes of signalling, in a manner such that these countermeasures can be initiated with components of the apparatus by means of the control signal of the control module, wherein a component for initiating a countermeasure is a gas flow device configured for the production of a flow of gas in the processing area, wherein the gas flow device comprises an inlet valve and an outlet valve, wherein the control signal is configured in a manner such that the inlet valve and the outlet valve are open,
wherein the apparatus comprises a gas channel behind the outlet valve and the sensor module of the sensor arrangement is disposed in this gas channel in a manner such that gas flowing through the gas channel serves as the gas sample for a measurement.

8. A measurement method with a sensor arrangement of claim 1, comprising the steps of:
generating a sensor signal by means of the sensor module of the sensor arrangement,
comparing the sensor signal or a variable derived from the sensor signal with a specified threshold value and determining whether the sensor module is measuring outside a predetermined action range,
if the comparison reveals that the sensor module is measuring outside a predetermined action range: initiating a predetermined countermeasure which is intended to modify the conditions in the apparatus in a manner such that the sensor module is again measuring in the action range, wherein a control signal is produced with which the countermeasure can be initiated.

9. The measurement method of claim 8, wherein in the context of the comparison of the sensor signal or a variable derived from the sensor signal with the specified threshold value, the following is determined:
whether the sensor signal lies over or under a threshold value which characterizes the threshold of the action range,
whether the variable derived from the sensor signal lies over or under the threshold value, wherein the variable is the first derivative with respect to time,
whether a maximum or minimum of the sensor signal as the threshold value has been passed,
whether the sensor signal lies under or over a first threshold value and the variable derived from the sensor signal lies under or over a second threshold value,
wherein the comparison is additionally carried out on the basis of a plurality of other measured values, with a plurality of measured values from the group formed by measured moisture content, a further sensor signal, measured temperature and measured pressure.

10. The measurement method of claim 9, wherein
the threshold value and the predetermined action range are determined and whether the sensor module is measuring within the action range is checked, and/or
a measuring curve is generated from a plurality of sensor signals at different times and a local maximum or minimum of the measuring curve is determined and this is used as the measure regarding whether the sensor signal lies beyond the threshold value, and/or
a maximum or minimum value for a standard deviation of the sensor signal is specified as the threshold value,
the manufacturing process which was selected is checked and the threshold value and therefore the action range is determined as a function of this manufacturing process, based on a total irradiation period per layer and/or a quantity of the build material which is used.

11. The measurement method of claim 8 wherein, for a reference measurement, the partial pressure of oxygen in the sensor module is reduced to a specified minimum density, by pumping with a voltage or an oxygen adsorbent or by specific flushing of a sample gas.

12. The measurement method of claim 8, wherein a reference measurement is carried out with the sensor module at another operating voltage, wherein the other preadjusted settings are selected in a manner such that a signal which is based on hydrogen and/or water vapour is suppressed and a ratio of oxygen to hydrogen and/or water vapour is determined on the basis of a reference signal obtained from this reference measurement and the sensor signal, wherein a correction of the sensor signal is carried out on the basis of the determined ratio.

13. The measurement method of claim 8, wherein one or more measures from the following group is selected as the countermeasure:
increasing the power of a pump for letting in a protective gas or inert gas,
opening an inlet valve and/or and outlet valve of the apparatus,
simulating a sensor signal,
introducing oxygen into the processing area,
removing water vapour and/or hydrogen from the processing area using a filter unit,
reducing the sensitivity to water and/or the sensitivity to hydrogen of the sensor module,
changing the sensor module for other measurements of a sensor signal,
using a second sensor, which is disposed at another position, for the measurement of a further sensor signal, wherein the comparison is additionally based on a signal ratio of the sensor signal to the further sensor signal,
changing the manufacturing mode of the apparatus in the form of an interruption,
changing the detection mode of the sensor module by applying another voltage or another temperature, or reversing the polarity of the electrodes, in the context of a reference measurement,
reversing the polarity of the sensor module,
changing a mode for the control of the inflow of inert gas.

14. The measurement method of claim 8, wherein a plurality of action ranges are defined with a respective threshold value and when one of these threshold values is crossed, one of the countermeasures associated with the respective threshold value is initiated.
